(19) United States
(12) Patent Application Publication
(10) Pub. No.: US 2009/0250637 A1
(43) Pub. Date: Oct. 8, 2009

(54) System and methods for filtering out-of-band radiation in EUV exposure tools

(75) Inventors: Robert P. Akins, Escondido, CA (US); Igor V. Fomenkov, San Diego, CA (US)

Correspondence Address: Matthew K. Hillman;CYMER, INC., Legal Dept., MS/4-2D, 17075 Thornmint Court, San Diego, CA, 92127-2413, US

(73) Assignee: CYMER, INC., San Diego, CA (US)

(21) Appl. No.: 12/384,171

(22) Filed: Mar. 31, 2009

Related U.S. Application Data
(60) Provisional application No. 61/072,855, filed on Apr. 2, 2008.

(51) Int. Cl. A61N 5/00; G01J 3/10

(52) U.S. Cl. 250/492.1; 250/504 R

(57) ABSTRACT

In a first aspect, an apparatus for exposing a substrate with EUV radiation is described herein which may comprise a target material; a laser source generating a laser beam having a wavelength, λ, for irradiating the target material to generate EUV radiation, the laser beam defining a primary polarization direction; at least one mirror reflecting the EUV radiation along a path to the substrate; and a polarization filter disposed along the path filtering at least a portion of light having the wavelength, λ.

### CROSS-REFERENCES TO RELATED APPLICATIONS

The present application claims priority to provisional U.S. application 61/072,855 filed Apr. 2, 2008, titled, “Systems and Methods for Filtering Out-of-Band Radiation in EUV Exposure Tools”.

The present application is related to co-pending U.S. patent application Ser. No. 12/004,905 filed on Dec. 20, 2007, entitled DRIVE LASER FOR EUV LIGHT SOURCE, Attorney Docket Number 2006-0065-01, co-pending U.S. patent application Ser. No. 11/827,803 filed on Jul. 13, 2007, entitled LASER PRODUCED PLASMA EUV LIGHT SOURCE HAVING A DROPLET STREAM PRODUCED USING A MODULATED DISTURBANCE WAVE, Attorney Docket Number 2007-0030-01, co-pending U.S. patent application Ser. No. 11/358,988 filed on Feb. 21, 2006, entitled LASER PRODUCED PLASMA EUV LIGHT SOURCE WITH PRE-PULSE, Attorney Docket Number 2005-0085-01, co-pending U.S. patent application Ser. No. 11/067,124 filed on Feb. 25, 2005, entitled METHOD AND APPARATUS FOR EUV PLASMA SOURCE TARGET DELIVERY, Attorney Docket Number 2004-0008-01, co-pending U.S. patent application Ser. No. 11/174,443 filed on Jun. 29, 2005, entitled LPP EUV PLASMA SOURCE MATERIAL TARGET DELIVERY SYSTEM, Attorney Docket Number 2005-0003-01, co-pending U.S. SOURCE MATERIAL DISPENSER FOR EUV LIGHT SOURCE, Attorney Docket Number 2005-0102-01, co-pending U.S. patent application Ser. No. 11/358,992 filed on Feb. 21, 2006, entitled LASER PRODUCED PLASMA EUV LIGHT SOURCE, Attorney Docket Number 2005-0081-01, co-pending U.S. patent application Ser. No. 11/174,299 filed on Jun. 29, 2005, and entitled, LPP EUV LIGHT SOURCE DRIVE LASER SYSTEM, Attorney Docket Number 2005-0044-01, co-pending U.S. patent application Ser. No. 11/406,216 filed on Apr. 17, 2006 entitled ALTERNATIVE FUELS FOR EUV LIGHT SOURCE, Attorney Docket Number 2006-0003-01, co-pending U.S. patent application Ser. No. 11/580,414 filed on Oct. 13, 2006, entitled, DRIVE LASER DELIVERY SYSTEMS FOR EUV LIGHT SOURCE, Attorney Docket Number 2006-0025-01, and co-pending U.S. patent application Ser. No. 11/644,153 filed on Dec. 22, 2006, entitled, LASER PRODUCED PLASMA EUV LIGHT SOURCE, Attorney Docket Number 2006-006-01, co-pending U.S. patent application Ser. No. 11/505,177 filed on Aug. 16, 2006, entitled EUV OPTICS, Attorney Docket Number 2006-0027-01, co-pending U.S. patent application Ser. No. 11/452,558 filed on Jun. 14, 2006 entitled DRIVE LASER FOR EUV LIGHT SOURCE, Attorney Docket Number 2006-0001-01, co-pending U.S. Pat. No. 6,928,093, issued to Webb, et al. on Aug. 9, 2005, entitled LONG DELAY AND HIGH TIS PULSE STRETCHER, U.S. application Ser. No. 11/394,512, Attorney Docket Number 2004-0144-01 filed on Mar. 31, 2006, and titled CONFOCAL PULSE STRETCHER, U.S. application Ser. No. 11/138,001 (Attorney Docket Number 2004-0128-01) filed on May 26, 2005, and titled SYSTEMS AND METHODS FOR IMPLEMENTING AN INTERACTION BETWEEN A LASER SHAPED AS A LINE BEAM AND A FILM DEPOSITED ON A SUBSTRATE, and U.S. application Ser. No. 10/141,216, filed on May 7, 2002, now U.S. Pat. No. 6,693,939, and titled, LASER LITHOGRAPHY LIGHT SOURCE WITH BEAM DELIVERY, U.S. Pat. No. 6,625,191, issued to Knowles et al., on Sep. 23, 2003, entitled VERY NARROW BAND, TWO CHAMBER, HIGH REP RATE GAS DISCHARGE LASER SYSTEM, U.S. application Ser. No. 10/012,002, Attorney Docket Number 2001-0090-01, U.S. Pat. No. 6,549,551 issued to Ness et al on Apr. 15, 2003 entitled INJECTION SEEDED LASER WITH PRECISE TIMING CONTROL, U.S. application Ser. No. 09/848,043, Attorney Docket Number 2001-0020-01 and U.S. Pat. No. 6,567,450, issued to Myers et al., on May 20, 2003, entitled VERY NARROW BAND, TWO CHAMBER, HIGH REP RATE GAS DISCHARGE LASER SYSTEM, U.S. application Ser. No. 09/943,343, Attorney Docket Number 2001-0084-01, co-pending U.S. patent application Ser. No. 11/509,925 filed on Aug. 25, 2006, entitled SOURCE MATERIAL COLLECTION UNIT FOR A LASER PRODUCED PLASMA EUV LIGHT SOURCE, Attorney Docket Number 2005-0086-01, the entire contents of each of which are hereby incorporated by reference herein.

### FIELD

The present application relates to extreme ultraviolet (“EUV”) exposure tools, e.g. steppers, scanners, etc., which produce, pattern and direct EUV light onto a substrate, e.g. a silicon wafer coated with a light sensitive material.

### BACKGROUND

Extreme ultraviolet (“EUV”) light, e.g., electromagnetic radiation having wavelengths of around 5-100 nm or less (also sometimes referred to as soft x-rays), and including light at a wavelength of about 13 nm, can be used in exposure processes, e.g. lithography, to produce extremely small features in substrates, e.g. silicon wafers.

Methods to produce EUV light include, but are not necessarily limited to, converting a material into a plasma state that has an element, e.g., xenon, lithium, tin, etc., with an emission line in the EUV range. In one such method, often termed laser produced plasma (“LPP”) the required plasma can be produced by irradiating a target material, for example, in the form of a droplet, stream or cluster of material, with one or more laser pulses, e.g. a pre-pulse and a relatively high power “main” pulse. For this purpose, CO₂ lasers, e.g., outputting light at infrared wavelengths, e.g. 9.3 μm or 10.6 μm, may present certain advantages when used as a so-called “drive laser” to irradiate a target material in an LPP process. This may be especially true for certain target materials, such as target materials containing tin. For example, one advantage may include the ability to produce a relatively high conversion efficiency between the drive laser input power and the output EUV power. Another advantage of CO₂ drive lasers may include the ability of the relatively long wavelength light (for example, as compared to deep UV at 193 nm) to reflect from relatively rough surfaces such as a reflective optic that has been coated with tin debris. This property of 10.6 μm radiation may allow reflective mirrors to be employed near the plasma, and in some cases within the same chamber as the plasma, for beam steering, focusing and/or adjusting the focal power of the drive laser beam.

In one particular arrangement, the EUV light from the plasma may be collected and directed to an intermediate focus, and thereafter conditioned, patterned by a patterning device, and then projected onto a substrate, e.g., resist coated wafer. During this process, the EUV light is typically reflected from a plurality of surfaces such as near-normal incidence mirrors, grazing incidence mirrors, reflective masks, etc., between the plasma and substrate, with each reflection resulting in a substantial loss in EUV light intensity (typically around 25-40% per reflection).

As used herein, the term “patterning device” should be broadly interpreted as referring to any means that can be used to endow an incoming radiation beam with a patterned cross-section, corresponding to a pattern that is to be created in a target portion of a substrate. Generally, the pattern will correspond to a particular functional layer in a device being created in the target portion, such as an integrated circuit or other device. Functionally, the placement of such a mask in the radiation beam causes selective reflection (in the case of a reflective mask) of the radiation impinging on the mask, according to the pattern on the mask.

Unfortunately, out-of-band radiation generated by the light source, i.e., radiation having wavelengths outside the desired band (e.g., 13.4 nm +/−2%) may also be reflected along the path described above and reach the substrate. This out-of-band radiation, which can include light generated by the plasma, e.g., deep UV, etc., as well as light from the drive laser, (i.e., infrared when a CO drive laser is used), may cause unwanted exposure of the light-sensitive resist and/or may undesirably heat reflective surfaces in the exposure system.

Along these lines, US 2002/0186811A1, which published on Dec. 12, 2002, discloses a grating element and diaphragm arrangement in which the grating is positioned in a beam path between the light source plasma and intermediate focus to spectrally filter light source radiation. In addition to adding an extra element and its concomitant EUV intensity losses, the grating, when positioned as disclosed, may be exposed to debris, e.g., ions, vapor, etc., from the plasma resulting in decreased efficiency, downtime, etc.

With the above in mind, applicants disclose systems and methods for filtering out-of-band radiation in EUV lithography tools.

### SUMMARY

In a first aspect, an apparatus for exposing a substrate with EUV radiation is described herein which may comprise a target material; a laser source generating a laser beam having a wavelength, λ, for irradiating the target material to generate EUV radiation, the laser beam defining a primary polarization direction; at least one mirror reflecting the EUV radiation along a path to the substrate; and a polarization filter disposed along the path filtering at least a portion of light having the wavelength, λ.

In one implementation of this aspect, the polarization filter may comprise a wire grid polarizer, and in a particular implementation, the wire grid polarizer may be a free-standing wire grid polarizer.

In one arrangement, the wire grid polarizer may comprise a plurality of wires, each wire aligned parallel to the primary polarization direction.

In a particular embodiment of this aspect, the wire grid may have a wire spacing period, p, with p<0.6λ.

In one setup, the at least one mirror may comprise a near-normal incidence, EUV reflector having a surface, the surface being a portion of a rotated ellipse.

For the apparatus, the laser source may have a laser gain medium comprising CO₂ gas.

In another aspect, an apparatus for exposing a substrate with EUV radiation is described herein which may comprise a target material; a laser source generating a laser beam having a wavelength, λ, for irradiating the target material to generate EUV radiation; and a patterning device having a surface imparting a pattern to the EUV radiation upon reflection therefrom, the patterning device further comprising a plurality of spaced-apart features, the features establishing a grating for diffracting at least a portion of light of wavelength, λ, incident upon the patterning device.

In one arrangement of this aspect, the features may be established to diffract at least fifty percent of the light of wavelength, λ into non-zero diffraction orders.

In a particular embodiment of this aspect, the patterning device may comprise an absorber layer overlaying a near-normal incidence EUV reflective multilayer coating and the features may constitute removed portions of the absorber layer, and in another embodiment, the patterning device may comprise an absorber layer overlaying a near-normal incidence EUV reflective multilayer coating and the features may constitute un-removed portions of the absorber layer.

In one setup of this aspect, the features may be spaced apart at a distance, d, with d<λ.

For the apparatus, the laser source has a laser gain medium comprising CO₂ gas.

In another aspect, an apparatus for exposing a substrate with EUV radiation is described herein which may comprise a target material; a laser source generating a laser beam having a wavelength, λ, for irradiating the target material to generate EUV radiation, at least one mirror reflecting the EUV radiation along a path to the substrate; and a free-standing wire grid disposed along the path filtering at least a portion of light having the wavelength, λ.

In one implementation of this aspect, the laser beam may define a primary polarization direction and the free-standing wire grid may be a wire grid polarizer.

In one arrangement, the wire grid may have a wire spacing period, p, with p<0.6λ.

In a particular embodiment of this aspect, the laser beam may be circularly polarized and the free-standing wire grid may be a first wire grid polarizer having a first polarizer transmission axis and the apparatus may further comprise a second wire grid polarizer having a second polarizer transmission axis, the second polarizer transmission axis being aligned orthogonal to the first polarizer transmission axis.

In one setup of this aspect, the free-standing wire grid may be configured to diffract at least twenty-five percent of the light of wavelength, λ, into non-zero diffraction orders.

For the apparatus, the laser source may have a laser gain medium comprising CO₂ gas.

In one embodiment, the at least one mirror may comprise a near-normal incidence, EUV reflector having a surface, the surface being a portion of a rotated ellipse.

### BRIEF DESCRIPTION OF THE DRAWINGS

FIG. 1 shows a simplified schematic view of an apparatus for exposing a substrate with EUV light according to an aspect of the present disclosure;

FIG. 2 shows a schematic, simplified view of four mirror projection system for use in the apparatus 10 shown in FIG. 1;

FIG. 3 shows a schematic, simplified view of an EUV light source, e.g., a laser-produced-plasma EUV light source for use in the apparatus 10 shown in FIG. 1;

FIG. 4 shows selected portions of an embodiment of a lithographic apparatus for exposing a substrate (such as a resist coated silicon wafer) with a patterned beam of EUV light, the apparatus having a system for filtering out-of-band radiation;

FIG. 5 shows a sectional view of a free-standing wire grid polarizer as seen along line 5-5 in FIG. 4;

FIG. 6 shows selected portions of another embodiment of a lithographic apparatus for exposing a substrate (such as a resist coated silicon wafer) with a patterned beam of EUV light, the apparatus having a system for filtering out-of-band radiation;

FIG. 7 shows a sectional view of a free-standing wire grid polarizer as seen along line 7-7 in FIG. 6;

FIG. 8 shows selected portions of another embodiment of a lithographic apparatus for exposing a substrate (such as a resist coated silicon wafer) with a patterned beam of EUV light, the apparatus having a system for filtering out-of-band radiation;

FIG. 9 shows a sectional view of a free-standing wire grid as seen along line 9-9 in FIG. 8;

FIG. 10 shows a section thru a reflective EUV mask blank;

FIG. 11 shows a section thru a reflective EUV patterning device;

FIG. 12 shows a top plan view of a reflective EUV patterning device; and

FIG. 13 shows a section thru a reflective EUV patterning device.

### DETAILED DESCRIPTION

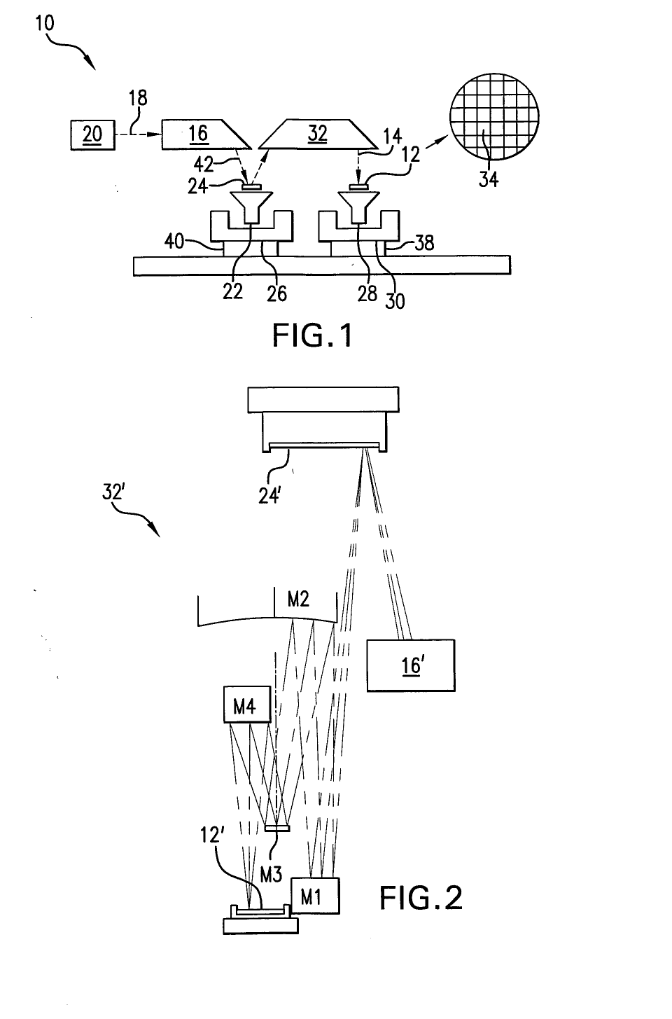

FIG. 1 schematically illustrates a lithographic apparatus (generally designated 10) for exposing a substrate 12 (such as a resist coated silicon wafer) with a patterned beam 14 of EUV light. In overview, the apparatus 10 may include an illumination system 16 configured to condition a radiation beam 18 from an EUV light source 20 and a patterning device support 22 (e.g., a mask table) constructed to support a patterning device 24 (e.g., a mask/reticle for endowing an incident radiation beam with a selected pattern in its cross-section). As further shown, the patterning device support 22 may be operably coupled to a first positioning unit 26 configured to accurately position the patterning device 24 and the apparatus 10 may also include a substrate table 28 constructed to support a substrate 12 (e.g., a resist-coated wafer) which is operably coupled to a second positioning unit 30 configured to accurately position the substrate 12. FIG. 1 also shows that a projection system 32 (e.g., a reflective projection system) may be provided that is configured to project the patterned EUV beam onto a target portion 34 (e.g., comprising one or more dies) of the substrate 12.

In more detail, for the apparatus 10, the illumination system 16 may include various types of optical components, such as reflective, diffractive, magnetic, electromagnetic, electrostatic or other types of optical components, or any combination thereof, for directing radiation, shaping radiation, controlling radiation and/or altering the intensity profile of the radiation beam.

As further shown in FIG. 1, light from the illumination system 16 may be made incident upon the patterning device 24 which imparts a pattern onto the radiation beam's cross-section. It should be noted that the pattern imparted to the radiation beam may not exactly correspond to the desired pattern in the target portion of the substrate 12, for example, the pattern may include phase-shifting features or so-called assist features.

From the patterning device 24, the beam may pass through a projection system 32 which reduces the pattern image and directs the beam onto a portion of the substrate 12. FIG. 2 illustrates a reflective projection system 32′ for illuminating a substrate 12′ with EUV light. As seen there, EUV light from illumination system 16′ reflects from the patterning device 24′ and thereafter enters the four mirror projection system 32′, reflecting from, in order, mirrors M1, M2, M3 and M4, and subsequently illuminating the substrate 12′. Although a four mirror projection system 32 is shown, it is to be appreciated that more or less than four mirrors may be used, for example, five and six mirror designs having increased numerical aperture (NA) have been previously suggested.

Referring back to FIG. 1, it can be seen that the positioning unit 30 may cooperate with a position sensor 38 (e.g. an interferometric device, linear encoder or capacitive sensor, etc.), to accurately move the substrate table 28, e.g., so as to position different target portions 34 in the path of the radiation beam 14. Similarly, the positioning unit 26 may cooperate with a position sensor 40 to accurately move the patterning device 24 relative to the beam 42 from the illumination system 16. With this arrangement, the apparatus 10 may be operated in one or more modes, such as step mode, scan mode, step and scan mode, etc.

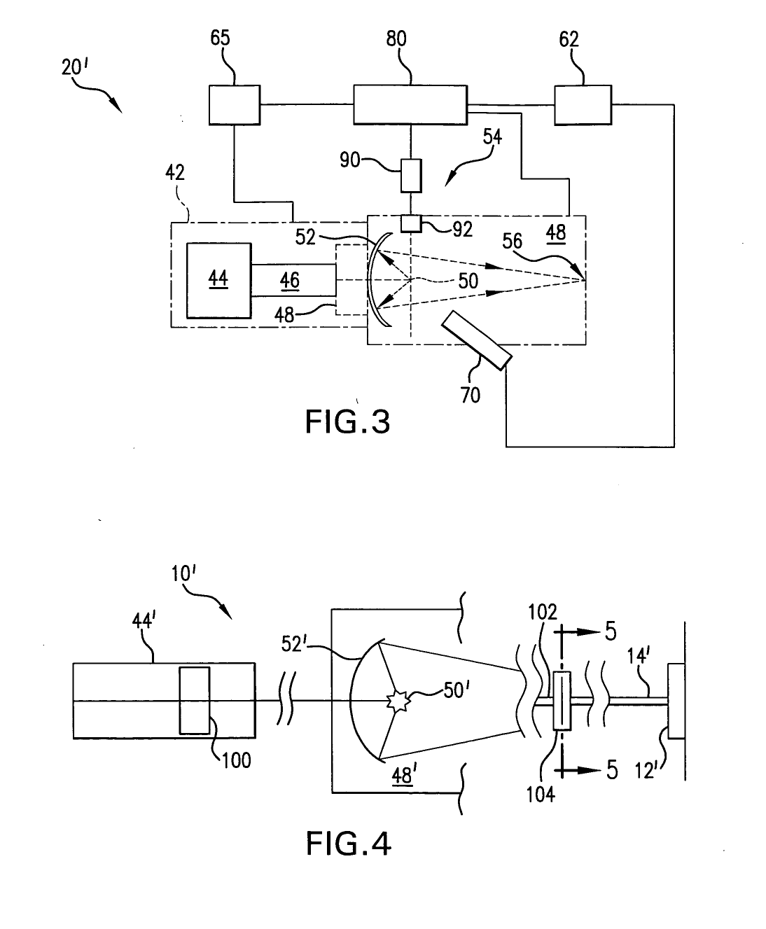

FIG. 3 shows a schematic, simplified view of an EUV light source, e.g., a laser-produced-plasma, EUV light source 20′, for use in the apparatus 10 shown in FIG. 1 and described above. As shown in FIG. 3, the LPP light source 20′ may include a system 42 for generating and delivering a train of light pulses. As shown, the system 42 may include a device 44 generating pulses (which in some cases may include one or more main pulses and one or more pre-pulses), an optional isolator 46 (shown with dashed lines to indicate an optional component) for isolating the device 44 from at least some downstream reflections, and an optional beam delivery system 48 (shown with dashed lines to indicate an optional component) for pulse shaping, focusing, steering and/or adjusting the focal power of the pulses exiting the isolator 46, and delivering the light pulses to a target location in chamber 48. For the EUV light source 20′, each light pulse may travel along a beam path from the system 42 and into the chamber 48 to illuminate a respective target droplet at an irradiation region, e.g., at or near a focus 50 of a mirror 52 having a reflective surface, the surface being a surface of revolution, e.g., an ellipse defining two focal points rotated about an axis passing through the two focal points.

Device 44 may include one or more lasers and/or lamps for providing one or more main pulses and, in some cases, one or more pre-pulses. Suitable lasers for use in the device 14 shown in FIG. 1 may include a pulsed laser device, e.g., a pulsed, gas-discharge CO₂ laser device producing radiation at 9.3 μm or 10.6 μm, e.g., with DC or RF excitation, operating at relatively high power, e.g., 10 kW or higher and high pulse repetition rate, e.g., 20 kHz or more. In one particular implementation, the laser may be an RF-pumped CO₂ laser having a MOPA configuration with multiple stages of amplification and having a seed pulse that is initiated by a Q-switched Master Oscillator (MO) with relatively low pulse energy and high repetition rate, e.g., 20-100 kHz. From the MO, the laser pulse may then be amplified, shaped, steered and/or focused before entering the LPP chamber 48. Continuously RF pumped, fast axial flow, CO₂ amplifiers may be used for the system 42. Alternatively, the RF pump may be pulsed. For example, a suitable CO₂ laser device having an oscillator and three amplifiers (O-PA1-PA2-PA3 configuration) is disclosed in co-pending U.S. patent application Ser. No. 11/174,299 filed on Jun. 29, 2005, and entitled, LPP EUV LIGHT SOURCE DRIVE LASER SYSTEM, Attorney Docket Number 2005-0044-01, the entire contents of which have been previously incorporated by reference herein. Alternatively, the laser may be configured as a so-called “self-targeting” laser system in which the droplet serves as one mirror of the laser's optical cavity. In some “self-targeting” arrangements, a master oscillator may not be required. Self-targeting laser systems are disclosed and claimed in co-pending U.S. patent application Ser. No. 11/580,414 filed on Oct. 13, 2006, entitled, DRIVE LASER DELIVERY SYSTEMS FOR EUV LIGHT SOURCE, Attorney Docket Number 2006-0025-01, the entire contents of which are hereby incorporated by reference herein.

Depending on the application, other types of lasers may also be suitable for use in the EUV light source 20′, e.g., an excimer or molecular fluorine laser operating at high power and high pulse repetition rate. Other examples include, a solid state laser such as Nd:YAG, e.g., having a slab, rod, fiber or disk shaped active media, a MOPA configured excimer laser system, e.g., as shown in U.S. Pat. Nos. 6,625,191, 6,549,551, and 6,567,450, an excimer laser having one or more chambers, e.g., an oscillator chamber and one or more amplifying chambers (with the amplifying chambers in parallel or in series), a master oscillator/power oscillator (MOPO) arrangement, a power oscillator/power amplifier (POPA) arrangement, a master oscillator/power ring amplifier (MOPRA), or a solid state laser that seeds one or more excimer or molecular fluorine amplifier or oscillator chambers, may be suitable. Other designs are possible.

A suitable beam delivery system 48 for pulse shaping, focusing, steering and/or adjusting the focal power of the pulses is disclosed in co-pending U.S. patent application Ser. No. 11/358,992 filed on Feb. 21, 2006, entitled LASER PRODUCED PLASMA EUV LIGHT SOURCE, Attorney Docket Number 2005-0081-01, the contents of which are hereby incorporated by reference herein. As disclosed therein, one or more beam delivery system optics may be in fluid communication with the chamber 48. Pulse shaping may include adjusting pulse duration, using, for example a pulse stretcher and/or pulse trimming.

As further shown in FIG. 3, the EUV light source 20′ may also include a target material delivery system 54, e.g., delivering droplets of a target material into the interior of a chamber 48 to the irradiation region where the droplets will interact with one or more light pulses, e.g., zero, one or more pre-pulses and thereafter one or more main pulses, to ultimately produce a plasma and generate an EUV emission. The target material may include, but is not necessarily limited to, a material that includes tin, lithium, xenon or combinations thereof. The EUV emitting element, e.g., tin, lithium, xenon, etc., may be in the form of liquid droplets and/or solid particles contained within liquid droplets. For example, the element tin may be used as pure tin, as a tin compound, e.g., SnBr₄, SnBr₂, SnH₄, as a tin alloy, e.g., tin-gallium alloys, tin-indium alloys, tin-indium-gallium alloys, or a combination thereof. Depending on the material used, the target material may be presented to the irradiation region at various temperatures including room temperature or near room temperature (e.g., tin alloys, SnBr₄) at an elevated temperature, (e.g., pure tin) or at temperatures below room temperature, (e.g., SnH₄), and in some cases, can be relatively volatile, e.g., SnBr₄. More details concerning the use of these materials in an LPP EUV source is provided in co-pending U.S. patent application Ser. No. 11/406,216 filed on Apr. 17, 2006, entitled ALTERNATIVE FUELS FOR EUV LIGHT SOURCE, Attorney Docket Number 2006-0003-01, the contents of which have been previously incorporated by reference herein.

Continuing with FIG. 3, the EUV light source 20′ may also include an optic 52, e.g., a collector mirror in the shape of a rotated ellipse (as described above) having, e.g., a silicon substrate and a graded multi-layer coating with alternating layers of Molybdenum and Silicon. FIG. 3 shows that the optic 52 may be formed with an aperture to allow the light pulses generated by the system 42 to pass through and reach the irradiation region. As shown, the optic 52 may have a reflective surface shaped as a rotated ellipse that has a first focus within or near the irradiation region and a second focus at a so-called intermediate region 56 where the EUV light may be output from the EUV light source 20′ and input to a device utilizing EUV light, e.g., an integrated circuit lithography tool (not shown). It is to be appreciated that other optics may be used in place of the rotated-ellipse shaped mirror for collecting and directing light to an intermediate location for subsequent delivery to a device utilizing EUV light, for example, the optic may be a rotated-parabola shaped mirror or may be configured to deliver a beam having a ring-shaped cross-section to an intermediate location, see e.g., co-pending U.S. patent application Ser. No. 11/505,177 filed on Aug. 16, 2006, entitled EUV OPTICS, Attorney Docket Number 2006-0027-01, the contents of which are hereby incorporated by reference.

Continuing with reference to FIG. 3, the EUV light source 20′ may also include an EUV controller 60, which may also include a firing control system 65 for triggering one or more lamps and/or laser devices in the system 42 to thereby generate light pulses for delivery into the chamber 48. The EUV light source 20′ may also include a droplet position detection system which may include one or more droplet imagers 70 that provide an output indicative of the position of one or more droplets, e.g., relative to the irradiation region. The imager(s) 70 may provide this output to a droplet position detection feedback system 62, which can, e.g., compute a droplet position and trajectory, from which a droplet error can be computed, e.g., on a droplet-by-droplet basis, or on average. The droplet error may then be provided as an input to the controller 60, which can, for example, provide a position, direction and/or timing correction signal to the system 42 to control a source timing circuit and/or to control a beam position and shaping system, e.g., to change the location and/or focal power of the light pulses being delivered to the irradiation region in the chamber 48.

The EUV light source 20′ may include one or more EUV metrology instruments for measuring various properties of the EUV light generated by the source 20′. These properties may include, for example, intensity (e.g., total intensity or intensity within a particular spectral band), spectral bandwidth, polarization, beam position, pointing, etc. For the EUV light source 20′, the instrument(s) may be configured to operate while the downstream tool, e.g., photolithography scanner, is on-line, e.g., by sampling a portion of the EUV output, e.g., using a pickoff mirror or sampling “uncollected” EUV light, and/or may operate while the downstream tool, e.g., photolithography scanner, is off-line, for example, by measuring the entire EUV output of the EUV light source 20′.

As further shown in FIG. 3, the EUV light source 20′ may include a droplet control system 90, operable in response to a signal (which in some implementations may include the droplet error described above, or some quantity derived therefrom) from the controller 60, to e.g., modify the release point of the target material from a droplet source 92 and/or modify droplet formation timing, to correct for errors in the droplets arriving at the desired irradiation region and/or synchronize the generation of droplets with the pulsed laser system 42.

More details regarding various droplet dispenser configurations and their relative advantages may be found in co-pending U.S. patent application Ser. No. 11/827,803 filed on Jul. 13, 2007, entitled LASER PRODUCED PLASMA EUV LIGHT SOURCE HAVING A DROPLET STREAM PRODUCED USING A MODULATED DISTURBANCE WAVE, Attorney Docket Number 2007-0030-01, co-pending U.S. patent application Ser. No. 11/358,988 filed on Feb. 21, 2006, entitled LASER PRODUCED PLASMA EUV LIGHT SOURCE WITH PRE-PULSE, Attorney Docket Number 2005-0085-01, co-pending U.S. patent application Ser. No. 11/067,124 filed on Feb. 25, 2005, entitled METHOD AND APPARATUS FOR EUV PLASMA SOURCE TARGET DELIVERY, Attorney Docket Number 2004-0008-01 and co-pending U.S. patent application Ser. No. 11/174,443 filed on Jun. 29, 2005, entitled LPP EUV PLASMA SOURCE MATERIAL TARGET DELIVERY SYSTEM, Attorney Docket Number 2005-0003-01, the contents of each of which are hereby incorporated by reference.

FIG. 4 shows selected portions of an embodiment of a lithographic apparatus (generally designated 10′) for exposing a substrate 12′ (such as a resist coated silicon wafer) with a patterned beam 14′ of EUV light. As shown, the apparatus 10′ may include an EUV light source e.g., a laser-produced-plasma EUV light source having a device 44′, e.g. laser source, generating a train of light pulses for interaction with a target material at a location 50′ in a chamber 48′ to generate an EUV output. As described above, the pulse train generated by the device 44′ may include one or more main pulses having a wavelength, λ, and one or more pre-pulses, which may (or may not) have the same wavelength, λ, as the main pulse. For the device 44′, optic 100 may be employed such that light exiting the device 44′ has a primary polarization direction, e.g., is linearly polarized to a significant extent. For example, the optic 100 may include one or more polarizers, e.g., thin film polarizers, wire grid polarizers, one or more transparent optics inclined at or near Brewster's angle, e.g., so-called Brewster's windows, etc. For the apparatus 10′, the optic 100 may be located within a laser optical cavity and/or along a beam path between the laser cavity and the target location 50′. Moreover, the optic 100 may effectively polarize one or more pre-pulse(s), one or more main pulse(s), and/or both pre-pulse(s) and main pulse(s). Different optics may be used to polarize the pre-pulse(s) and main pulse(s) or a common optic may be employed to polarize pre-pulse(s) and main pulse(s).

Continuing with FIG. 4, it can be seen that linearly polarized light defining a primary polarization direction and having a wavelength, λ, may be made incident on a target material at the location 50 whereupon EUV light is emitted and the incident laser beam having wavelength, λ, is scattered. Light from the target location 50′ including EUV light and scattered light having wavelength, λ, will be reflected from mirror 52′ onto beam path 102 and some, most or all of the light having wavelength, λ, traveling along the beam path 102 may be linearly polarized in the primary polarization direction established by the optic 100. As shown, light traveling on beam path 102 reaches and exposes substrate 12′. FIG. 4 further shows that beam path 102 passes through wire grid 104 which filters at least some of the light having wavelength, λ, from the beam path 102. Cross-referencing FIGS. 1 and 4, it can be seen that light from the mirror 52, 52′ may pass through one or more optics modules such as an illumination system 16 (described above), patterning device 24 (described above) and/or projection system 32 (described above) along the beam path 102 from the mirror 52, 52′ to the substrate 12, 12′. For the apparatus shown in FIG. 4, the wire grid 104 may be positioned at various locations along the path 104. For example, the wire grid 104 may be positioned upstream of the illumination system 16, within the illumination system 16 (e.g., between two optics in the illumination system 16), between the illumination system 16 and the patterning device 24, between the patterning device 24 and projection system 32, within the projection system 32 (e.g. between two mirrors in the projection system 32), and/or between the projection system 32 and substrate 12. This variation in possible locations is illustrated in FIG. 4.

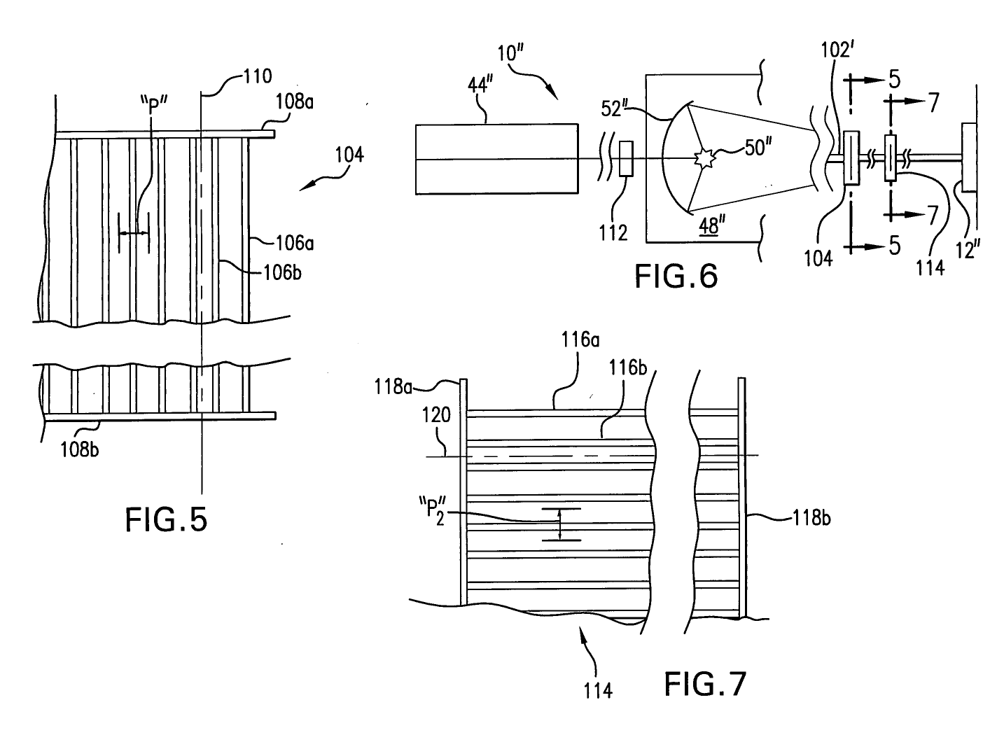

FIG. 5 illustrates a wire grid 104 in more detail. As shown, the wire grid 104 may be a free standing wire grid polarizer having a plurality of conductive wires 106a,b which are spaced-apart with each wire 106a,b attached to and suspended between a pair of spaced-apart supports 108a,b such that each wire 106a,b is maintained substantially parallel to the other wires and substantially parallel to a wire grid transmission axis 110. FIG. 5 further shows that the wires 106a,b are spaced-apart to establish wire spacing period, “p”, for the wire grid, with the distance “p” being measured from a first midpoint between two adjacent wires to the next midpoint between two adjacent wires, measured in a direction substantially orthogonal to the transmission axis 110, as shown.

For filtration of linearly polarized light having a wavelength, λ, a wire grid having a wire spacing period, p, with p<0.6λ may be used. For the case where the device 44′ includes a gain media comprising CO₂, and generating light having wavelength, λ, of 9.3 μm or 10.6 μm, a wire spacing period, p, of less than about 5.6 μm or less than about 6.4 μm, respectively, may be used. For these grids, wires having diameters of about 2 μm or less may be used. Smaller wire spacing periods, p, and/or thinner wires, e.g., submicron wires, may be employed to achieve greater filtering and/or to filter rays having an angle of incidence on the wire grid that deviates from zero degrees. For example, a wire spacing period, p, with p<0.2λ may be used.

In use, the wire grid 104 may be positioned in the beam path 102 and oriented, for example using a rotational mount, such that the wire grid transmission axis is substantially parallel to the primary polarization direction of the light having a wavelength, λ. Filtering of light having wavelength, λ, may be via reflection and/or absorption. Typically, the EUV light incident on the grid may have a reduced transmission intensity that is proportional to the wire fill factor.

FIG. 6 shows selected portions of another embodiment of a lithographic apparatus (generally designated 10″) for exposing a substrate 12″ (such as a resist coated silicon wafer) with a patterned beam 14″ of EUV light. As shown, the apparatus 10″ may include an EUV light source e.g., a laser-produced-plasma EUV light source having a device 44″, e.g., laser source, generating a train of light pulses for interaction with a target material at a location 50″ in a chamber 48″ to generate an EUV output. As described above, the pulse train generated by the device 44′ may include one or more main pulses having a wavelength, λ, and one or more pre-pulses, which may or may not have the same wavelength, λ, as the main pulse.

For the device 10″, an optical isolator 112 may be positioned along a beam path between the device 44″ and an irradiation site 50″ where a droplet will intersect with the beam path to isolate the gain media of the device 44″ from light reflected from the droplet (so-called back reflections). The isolator 112 may cooperate with a device 44″ having, e.g. polarizers and/or Brewster's windows and which outputs linear polarized light. For this case, the optical isolator 112 may include, for example, a phase retarder mirror which, when reflecting light, converts linear polarized light to circularly polarized light, and converts circularly polarized light to linear polarized light. Thus, light initially having a primary polarization direction that is subsequently reflected twice from the phase retarder mirror is rotated ninety degrees from the primary polarization direction, i.e., the twice-reflected light becomes linearly polarized in a direction orthogonal to the primary polarization direction. In addition to the phase retarder mirror, the isolator 112 may also include a linear polarization filter, e.g., isolator mirror which absorbs light that is linearly polarized in a direction orthogonal to the primary polarization direction. With this arrangement, light reflected on the beam path from the target material, e.g., droplet, is absorbed by the optical isolator 112 and cannot re-enter the device 44″. For example, a suitable unit for use with CO₂ lasers may be obtained from Kugler GmbH, Heiligenberger Str. 100, 88682, Salem, Germany, under the trade name Queller and/or “isolator box”. Typically, the optical isolator 112 functions to allow light to flow from the device 44″ to the droplet virtually unimpeded while allowing only about one percent of back-reflected light to leak through the optical isolator 112 and reach the device 44″.

With this arrangement, circularly polarized light having a wavelength, λ, is made incident on a target material at the location 50″ whereupon EUV light is emitted and the incident laser beam having wavelength, λ, is scattered. Light from the target location 50″ including EUV light and scattered light having wavelength, λ, will be reflected from mirror 52″ onto beam path 102′ and some, most or all of the light having wavelength, λ, traveling along the beam path 102′ may be circularly polarized. As shown, light traveling on beam path 102′ reaches and exposes substrate 12″. FIG. 6 further shows that beam path 102′ passes, in series, through wire grid 104 and wire grid 114 which together filter at least some of the light having wavelength, λ, from the beam path 102′. Cross-referencing FIGS. 1 and 6, it can be seen that light from the mirror 52, 52″ may pass through one or more optics modules such as an illumination system 16 (described above), patterning device 24 (described above) and/or projection system 32 (described above), along the beam path 102′ from the mirror 52, 52″ to the substrate 12, 12″. For the apparatus shown in FIG. 4, the wire grids 104, 114 may be positioned at various locations along the path 102′ with zero, one or more optical components/modules positioned between the two wire grids 104, 114.

Wire grid 104 is shown in FIG. 5 and described in detail above with reference to FIG. 5. As shown in FIG. 7, the wire grid 114 may also be a free standing wire grid polarizer having a plurality of conductive wires 116a,b which are spaced apart with each wire 116a,b attached to and suspended between a pair of spaced apart supports 118a,b such that each wire 116a,b is maintained substantially parallel to the other wires and substantially parallel to a wire grid transmission axis 120. FIG. 7 further shows that the wires 116a,b are spaced-apart to establish wire spacing period, “p₂”, for the wire grid 114, with the distance “p₂” being measured from a first midpoint between two adjacent wires to the next midpoint between two adjacent wires, measured in a direction substantially orthogonal to the transmission axis 120, as shown.

For filtration of (nonpolarized and/or circularly polarized light having a wavelength, λ, wire grids 104, 114, having wire spacing period, p, p₂ less than about 0.6λ may be used. For the case where the device 44′ includes a gain media comprising CO₂, and generating light having wavelength, λ, of 9.3 μm or 10.6 μm, a wire spacing periods, p and p₂ of less than about 5.6 μm (for λ=9.3 μm) or less than about 6.4 μm (for λ=10.6 μm), may be used. For these grids, wires having diameters of about 2 μm or less may be used. Smaller wire spacing periods, p, p₂ and/or thinner wires, e.g., submicron wires, may be employed to achieve greater filtering and/or to filter rays having an angle of incidence on the wire grid(s) that deviate from zero degrees. For example, wire spacing periods, p, p₂ less than about 0.2λ may be used.

In use, the wire grids 104, 114 may be positioned in the beam path 102′ and oriented, for example using rotational mounts, such that the wire grid transmission axis 110 for the grid 104 is substantially orthogonal to the wire grid transmission axis 120 for the grid 114, as shown in FIG. 6. Typically, EUV light may pass through the grid having a reduced transmission intensity proportional to the wire fill factor of the grid combination. Note also, that a second wire grid polarizer 114 may be employed in the embodiment shown in FIG. 5 to further filter light having wavelength, λ, which is not linearly polarized parallel to the primary polarization direction.

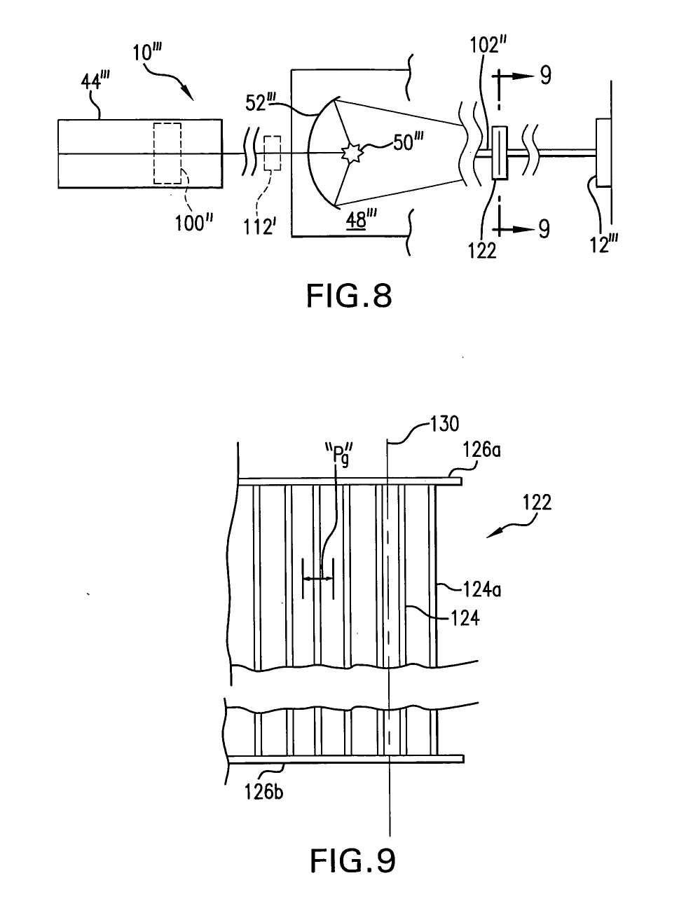

FIG. 8 shows selected portions of another embodiment of a lithographic apparatus (generally designated 10′″) for exposing a substrate 12′″ (such as a resist coated silicon wafer) with a patterned beam 14′″ of EUV light. As shown, the apparatus 10′″ may include an EUV light source e.g., a laser-produced-plasma EUV light source having a device 44′″, e.g. laser source, generating a train of light pulses for interaction with a target material at a location 50′″ in a chamber 48′″ to generate an EUV output. As described above, the pulse train generated by the device 44′″ may include one or more main pulses having a wavelength, λ, and one or more pre-pulses, which may or may not have the same wavelength, λ, as the main pulse.

For the apparatus 10′″, an optional optic 100″ may be employed such that light exiting the device 44′″ has a primary polarization direction, e.g., is linearly polarized to a significant extent (as described above) and/or an optional optical isolator 112′ (as described above) may be positioned along a beam path between the device 44″ and an irradiation site 50′ where a droplet will intersect with the beam path. Thus, depending on which of these optional components are employed, for the apparatus 10′″, light irradiating the target at location 50′″ may be non-polarized, linearly polarized or circularly polarized, and light scattered by the target material and reflected onto beam path 102″ by optic 52′″ may be non-polarized, linearly polarized or circularly polarized.

FIG. 8 further shows that light traveling on beam path 102″ reaches and exposes substrate 12″ after passing through wire grid 122 which filters at least some of the light having wavelength, λ, from the beam path 102″. As detailed further below, filtration by wire grid 122 may be accomplished via diffraction and may also include polarization filtering (via reflection and/or absorption as described above).

Cross-referencing FIGS. 1 and 8, it can be seen that light from the mirror 52, 52′″ may pass through one or more optics modules, such as an illumination system 16 (described above), patterning device 24 (described above) and/or projection system 32 (described above) along the beam path 102″ from the mirror 52, 52′″ to the substrate 12, 12′″. For the apparatus shown in FIG. 8, the wire grid 122 may be positioned at various locations along the path 102″. For example, the wire grid may be positioned upstream of the illumination system 16, within the illumination system 16 (e.g. between two optics in the illumination system 16), between the illumination system 16 and the patterning device 24, between the patterning device 24 and projection system 32, within the projection system 32 (e.g., between two mirrors in the projection system 32), and/or between the projection system 32 and substrate 12. This variation in possible locations is illustrated in FIG. 8.

FIG. 9 illustrates a wire grid 122 in more detail. As shown, the wire grid 122 may be a free-standing wire grid having a plurality of wires 124a,b, which may or may not be conductive, and which are spaced apart with each wire 124a,b attached to and suspended between a pair of spaced-apart supports 126a,b such that each wire 124a,b is maintained substantially parallel to the other wires and substantially parallel to a wire grid axis 130.

FIG. 9 further shows that the wires 124a,b are spaced apart to establish wire spacing period, “p_{g}”, for the wire grid, with the distance “p_{g}” being measured from a first midpoint between two adjacent wires to a second midpoint between two adjacent wires, in a direction substantially orthogonal to the wire grid axis 130, as shown. For the embodiment shown in FIGS. 8 and 9, wire spacing period, p_{g}, may be set relative to the wavelength, λ, such that a portion of light having wavelength, λ, incident upon the wire grid 122 is diffracted into nonzero diffraction orders and, as a consequence, is filtered from the beam path 102″. For example, diffraction of both s- and p-polarization generally occurs for p_{g}>0.5λ, (although not necessarily with equal efficiency). Moreover, for a wire grid 122 with conductive wires, a transition region 0.5λ<p_{g}<2λ may exist in which both diffraction and reflection/absorption of light polarized orthogonal to the wire grid axis 130. Thus, for the apparatus 10′″, the wire spacing period, p_{g}, may be set relative to the wavelength, λ, such that the wire grid operates as a filter in the transition region or the wire spacing period, p_{g}, may be set larger, e.g. p_{g}>2λ such that filtration is in large part due only to diffraction effects.

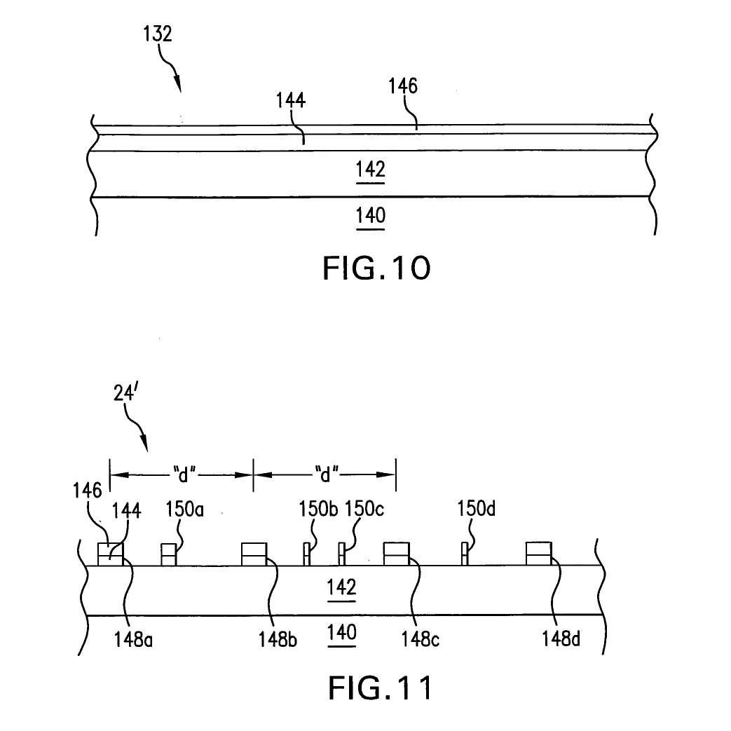

FIG. 10 shows a sectional view of a mask blank 132, which can be patterned to produce a patterning device 24′ (shown in FIG. 11) for use in the apparatus 10 shown in FIG. 1. As shown, the patterning device 24′ may be a reflective reticle/mask having a substrate 140 such as silicon or glass having an EUV reflective coating 142 formed thereon. The reflective coating 142 is typically a multilayer coating having 20-80 bi-layers, each bi-layer having a layer of relatively high index of refraction material and a layer of relatively low index of refraction material. For example, each bi-layer may include a layer of molybdenum and a layer of silicon. In some cases, the reflective coating 142 may also include a capping layer to protect the bi-layers. As further shown in FIG. 10, a buffer layer 144 (e.g., silicon dioxide) may be deposited to contact and overlay the reflective coating 142, and an absorber layer 146 composed of an EUV absorptive material (e.g., silver, tungsten, gold, tantalum, titanium, chromium, lead, polyimide, etc.) may be deposited to contact and overlay the buffer layer 144.

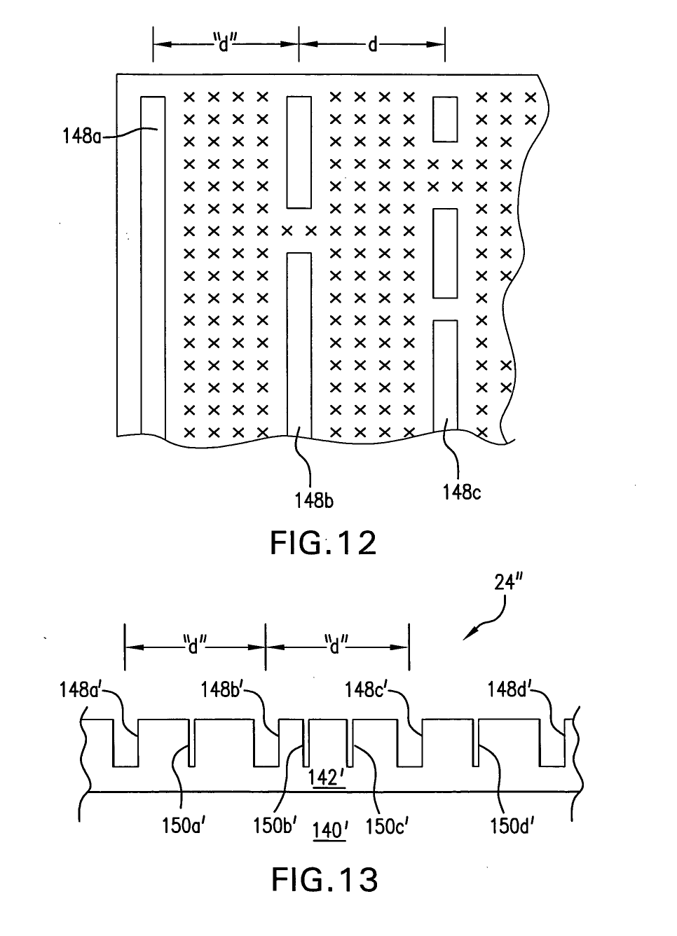

FIGS. 11 and 12 illustrate that the mask blank 132 shown in FIG. 10 may be patterned to produce a patterning device 24′ having elongated features 148a-d which are spaced from each other at a distance “d” for diffracting light having a wavelength, λ, (e.g. d≦λ) and representative features 150a-d which constitute a pattern for establishing a layer of an integrated circuit (IC) when the patterning device is used to expose a substrate 12 (see FIG. 1) with EUV light (note the X's in FIG. 12 represent the area on the patterning device in which features 150a-d may be placed). It is to be noted from FIG. 12 that the elongated features 148a-c do not necessarily extend the full length of the patterning device 24′, instead, as shown, one or more of the elongated features 148a-d may be segmented to allow the IC layer pattern to extend through gap established by the segmentation.

Features 148a-d, 150a-d may be patterned in the buffer layer 144 and absorbing layer 146 using, for example, a photolithographic process. In one technique, the mask blank 132 shown in FIG. 10 may be coated with a light sensitive layer, e.g., resist layer. Then, the resist layer may be exposed and developed. A first etching process may then be used to remove portions of the absorbing layer whereupon an inspection and repair (if necessary) of the pattern may be performed. With a suitable pattern in the absorbing layer 146, a second etching step may be performed to etch the pattern in the buffer layer 144, exposing the reflective coating 142.

FIG. 13 shows an alternate technique for producing a patterning device 24″ having elongated features 148a′-d′ which are spaced from each other at a distance “d” for diffracting light having a wavelength, λ, (e.g., d≦λ) and representative features 150a′-d′ which constitute a pattern for establishing a layer of an integrated circuit (IC) when the patterning device is used to expose a substrate 12 (see FIG. 1), with EUV light. As shown, the patterning device 24″ may be a reflective reticle/mask having a substrate 140′ such as silicon or glass having an EUV reflective coating 142′ formed thereon. The reflective coating 142′ is typically a multilayer coating having 20-80 bi-layers, each bi-layer having a layer of relatively high index of refraction material and a layer of relatively low index of refraction material. For example, each bi-layer may include a layer of molybdenum and a layer of silicon. In some cases, the reflective coating 142′ may also include a capping layer to protect the bi-layers. As further shown, features 148a′-d′, 150a′-d′ may be etched in the reflective coating 142′, reducing the coating's reflectivity, e.g., using a photolithography process. Alternatively, the reflectivity of the reflective coating 142′ may be selectively decreased to produce features using an ion beam to damage selected portions of the reflective coating 142′ (not shown).

As used herein, the term “optic” and its derivatives includes, but is not necessarily limited to, components which reflect and/or transmit and/or operate on incident light and includes, but is not limited to, lenses, windows, filters, wedges, prisms, grisms, gradings, transmission fibers, etalons, diffusers, homogenizers, detectors and other instrument components, input apertures, axicons and mirrors including multi-layer mirrors, near-normal incidence mirrors, grazing incidence mirrors, specular reflectors and diffuse reflectors. Moreover, as used herein, the term “optic” and its derivatives is not meant to be limited to components which operate solely or to advantage within one or more specific wavelength range(s) such as at the EUV output light wavelength, the irradiation laser wavelength, a wavelength suitable for metrology or some other wavelength, unless otherwise specified herein.

While the particular embodiment(s) described and illustrated in this patent application in the detail required to satisfy 35 U.S.C. §112 are fully capable of attaining one or more of the above-described purposes for, problems to be solved by, or any other reasons for, or objects of the embodiment(s) above described, it is to be understood by those skilled in the art that the above-described embodiment(s) are merely exemplary, illustrative and representative of the subject matter which is broadly contemplated by the present application. Reference to an element in the following Claims in the singular is not intended to mean, nor shall it mean in interpreting such Claim element “one and only one” unless explicitly so stated, but rather “one or more”. All structural and functional equivalents to any of the elements of the above-described embodiment(s) that are known or later come to be known to those of ordinary skill in the art, are expressly incorporated herein by reference and are intended to be encompassed by the present Claims. Any term used in the Specification and/or in the Claims and expressly given a meaning in the Specification and/or Claims in the present Application shall have that meaning, regardless of any dictionary or other commonly used meaning for such a term. It is not intended or necessary for a device or method discussed in the Specification as an embodiment to address or solve each and every problem discussed in this Application, for it to be encompassed by the present Claims. No element, component, or method step in the present disclosure is intended to be dedicated to the public regardless of whether the element, component, or method step is explicitly recited in the Claims. No claim element in the appended Claims is to be construed under the provisions of 35 U.S.C. §112, sixth paragraph, unless the element is expressly recited using the phrase “means for” or, in the case of a method claim, the element is recited as a “step” instead of an “act”.

I/We claim:

1. An apparatus for exposing a substrate with EUV radiation, said apparatus comprising:
a target material;
a laser source generating a laser beam having a wavelength, λ, for irradiating said target material to generate EUV radiation, said laser beam defining a primary polarization direction;
at least one mirror reflecting said EUV radiation along a path to the substrate; and
a polarization filter disposed along said path filtering at least a portion of light having said wavelength, λ.

2. An apparatus as recited in claim 1 wherein said polarization filter comprises a wire grid polarizer.

3. An apparatus as recited in claim 2 wherein said wire grid polarizer is a free-standing wire grid polarizer.

4. An apparatus as recited in claim 2 wherein said wire grid polarizer comprises a plurality of wires, each wire aligned parallel to said primary polarization direction.

5. An apparatus as recited in claim 4 wherein said wire grid has a wire spacing period, p, with p<0.6λ.

6. An apparatus as recited in claim 1 wherein said at least one mirror comprises a near-normal incidence, EUV reflector having a surface, the surface being a portion of a rotated ellipse.

7. An apparatus as recited in claim 1 wherein said laser source has a laser gain medium comprising CO₂ gas.

8. An apparatus for exposing a substrate with EUV radiation, said device comprising:
a target material;
a laser source generating a laser beam having a wavelength, λ, for irradiating said target material to generate EUV radiation; and
a patterning device having a surface imparting a pattern to the EUV radiation upon reflection therefrom, the patterning device further comprising a plurality of spaced apart features, the features establishing a grating for diffracting at least a portion of light of wavelength, λ incident upon the patterning device.

9. An apparatus as recited in claim 8 wherein the features diffract at least fifty percent of the light of wavelength, λ into non-zero diffraction orders.

10. An apparatus as recited in claim 8 wherein said patterning device comprises an absorber layer overlaying a near-normal incidence EUV reflective multilayer coating and said features constitute removed portions of said absorber layer.

11. An apparatus as recited in claim 8 wherein said patterning device comprises an absorber layer overlaying a near-normal incidence EUV reflective multilayer coating and said features constitute un-removed portions of said absorber layer.

12. An apparatus as recited in claim 8 wherein said features are spaced apart at a distance, d, with d<λ.

13. An apparatus as recited in claim 8 wherein said laser source has a laser gain medium comprising CO₂ gas.

14. An apparatus for exposing a substrate with EUV radiation, said apparatus comprising:
a target material;
a laser source generating a laser beam having a wavelength, λ, for irradiating said target material to generate EUV radiation, at least one mirror reflecting said EUV radiation along a path to the substrate; and
a free-standing wire grid disposed along said path filtering at least a portion of light having said wavelength, λ.

15. An apparatus as recited in claim 14 wherein said laser beam defines a primary polarization direction and said free-standing wire grid is a wire grid polarizer.

16. An apparatus as recited in claim 15 wherein said wire grid has a wire spacing period, p, with p<0.6λ.

17. An apparatus as recited in claim 14 wherein said laser beam is circularly polarized and said free-standing wire grid is a first wire grid polarizer having a first polarizer transmission axis and said apparatus further comprises a second wire grid polarizer having a second polarizer transmission axis, said second polarizer transmission axis being aligned orthogonal to said first polarizer transmission axis.

18. An apparatus as recited in claim 14 wherein said free-standing wire grid diffracts at least twenty-five percent of the light of wavelength, λ, into non-zero diffraction orders.

19. An apparatus as recited in claim 14 wherein said laser source has a laser gain medium comprising CO₂ gas.

20. An apparatus as recited in claim 14 wherein said at least one mirror comprises a near-normal incidence, EUV reflector having a surface, the surface being a portion of a rotated ellipse.
